(19) United States
(12) Patent Application Publication
(10) Pub. No.: US 2026/0090759 A1
(43) Pub. Date: Apr. 2, 2026

(54) METHOD OF USING A FLAVOUR AND AROMA GENERATION SYSTEM

(71) Applicant: Alan CHALMERS, Kenilworth (GB)

(72) Inventor: Alan CHALMERS, Kenilworth (GB)

(21) Appl. No.: 18/901,891

(22) Filed: Sep. 30, 2024

(51) Int. Cl. A61B 5/00; A61B 10/00; G01N 33/00

(52) U.S. Cl. CPC A61B 5/4017; A61B 5/4064; A61B 5/4088; G01N 33/0001; A61B 2010/0003

(57) ABSTRACT

A method of measuring an individual's taste and/or smell perception comprises a system to deliver to a user pluralities of artificial flavour sensations to be compared each comprising a plurality of taste and smell components selected from the group comprising sweetness, sourness, saltiness, bitterness, umami, astringency, capsaicin, oiliness, and aroma components. Each plurality contains at least one base flavour and one flavour to be compared against the base flavour. Delivery for the flavours can be through cups, vials, or other vessels. The system may identify a deviation of individual's flavour perception from that of a normal population's flavour perception distribution.

### FIELD OF THE INVENTION

This invention relates to a method of using a flavour generation system.

### BACKGROUND

US 2023/0051941 published 16 Feb. 2023 (incorporated by reference), there is described a system to deliver to a user artificial flavour sensations equivalent to a selected desired real flavour which has:
a. a database in which is stored data of a number of real flavours broken down into taste components;
b. containers containing taste components namely: sweetness, sourness, saltiness, bitterness, umami, astringency, capsaicin, oiliness;
c. a delivery means controlled from the database to provide to a user from the containers to a user taste sensation corresponding to the selected real flavour.

The database of US Patent 2023/0051941 may also contain information concerning smell, appearance and/or feel of a flavour.

In one embodiment, the delivery means comprises a cup or other vessel. This can deliver to a user an artificial flavour and, optionally, smell equivalent to a selected desired real flavour, and can be particularly useful in blending facilities (such as tea and coffee blending) and food manufacturing facilities. Also, containers containing smell components of a flavour can be provided, the smell components from the containers being delivered to the delivery means.

### SUMMARY OF INVENTION

According to the invention a method of indicating an individual's flavour perception comprises delivering to a user comparative taste sensations via a delivery means, the comparative taste sensations being pairs of artificial flavours to be compared by the individual, each member of each pair comprising a plurality of flavour components selected from the group comprising sweetness, sourness, saltiness, bitterness, umami, astringency, capsaicin, oiliness and aroma components associated with each flavour of the pair, one flavour within each pair being identical in each of the plurality of pairs, the other member of each pair being the same or different, and in which the outcome of the comparison by the individual is compared against a mean population response to the same comparative flavour sensations and in which deviation below the normal response pattern acts as an indication of the degradation of flavour sensation in the individual.

To minimize the impact of guessing on the part of the individual, normally, at least eight pairs of artificial flavours are compared by an individual.

The delivery means may comprise a plurality of pairs of vials one vial of a pair containing a flavour that is identical in at least one vial in each of the pairs and the other vial in a pair containing the flavour to be compared by the individual against the flavour that is identical in at least one vial in each of the pairs. The pairs of vials may be mounted in a test kit to be supplied to a user.

A method of the present invention can be used to identify the possible early onset of a neurodegenerative condition or possible brain injury in an individual.

### INTRODUCTION TO THE DRAWINGS

FIG. 1 illustrates the first stage of a method of measuring an individual's taste and/or smell perception;

FIG. 2 illustrates the second stage of the said method of measuring an individual's taste and/or smell perception.; and

FIG. 3 illustrates results obtained from the method of measuring an individual's taste and/or smell perception using the invention.

FIG. 4 is a schematic view of electronic machinery configured to implementation of the methods described herein.

### ILLUSTRATIVE EXAMPLES

A Virtual Flavour is an authentic simulation of a real flavour using UK Food Standards Agency approved food-safe chemicals. The virtual flavour is made up of individual components to simulate the real flavour. Currently the components are the following (although these can be added to in the future)

Six taste components (sweet, sour, salty, bitter1, bitter2, umami). Two bitter components are currently used as the human tongue is particularly sensitive to bitter tastes. There are three mouth feel components (astringency, capsaicin, oiliness) and three colour components (red, blue, yellow). In addition, ten aroma components are used. There are thousands of aroma components. However, for this invention the following major aromas are used: Citrus, floral, fruity, woody, earthy, nutty, phenolic, smoky, cocoa, anise.

Analysis of a real flavour provides the recipe for the virtual flavour, which is the amount of each component that is needed in the virtual flavour to accurately simulate the real flavour.

A Virtual Flavour (flavour A) and a comparator to that Virtual Flavour (Flavour B) are prepared and delivered by a dispenser as set out above.

A difference between one flavour and another (flavour A and flavour B) is the measured different amount in terms of one or more components of flavour A compared with flavour B. For example, Flavour A can contain more of the sweet component than Flavour B, or Flavour A may contain less citrus aroma and saltier component than Flavour B, etc.

An Always Noticeable Difference is the amount one or more of the components must be changed for a difference to be noticeable, detectable at 100% of the time.

A Just Noticeable Difference (JND) is the amount one or more of the components must be changed for a difference to be noticeable, detectable at least half the time.

Initially, as flavour perceptions can vary naturally by demographics information, such as age, sex, whether the person smokes, ethnicity, etc., a person (the user) on whom the method is to be deployed may be asked to provide such information.

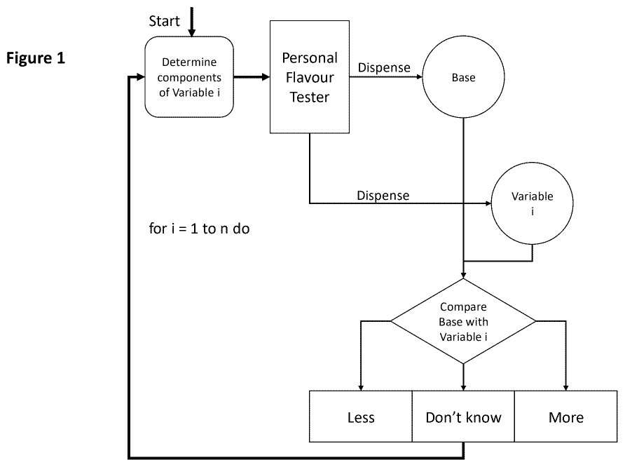

The initial step is shown in FIG. 1. The user of the Personal Flavour Tester device is dispensed user samples by the dispenser—a Base and a Variable i.

The user tries both the Base and Variable i. and is asked to state for one or more components whether:
a. it is not possible to distinguish any difference between the two samples;
b. the Variable i has more of the one or more components than the Base;
c. the Variable i has less of the one or more components than the Base

The Variable i is then placed in the chosen position on a grid with the three above categories and the result recorded. This may be repeated several times, each time the Base is unchanged, but the Variable may change times Variable i. ii, iii, . . . n.

TABLE 1

| | | |
|---|---|---|
| Less | Do not know | More |

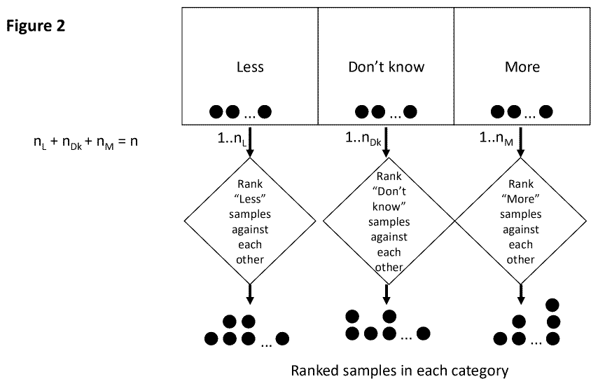

The outcomes in each category are examined and ranked in order of any perceived differences in one or more components (see FIG. 2). These results can be compared against previously prepared standards for the person's (user's) demographics, e.g., age group, sex, whether the person smokes, ethnicity, etc., with results outside the expected range identifying a potential issue needing further investigation.

In another approach eighty-nine people were given n a total of nine samples to taste and compare them. There was one BASE sample, and eight variable samples (e.g., Var1, Var2, . . . ) given to you one at a time to compare against the BASE.

Participants were asked to compare each Var sample to BASE sample and write in the table which one is sweeter BASE (B) or Var (V). If a participant could not tell, they were asked to the ‘can't tell” box.

TABLE 2

| | | |
|---|---|---|
| Variable | Which is Sweeter (B or V) | Can't Tell |

1
2
3
4
5
6
7
8

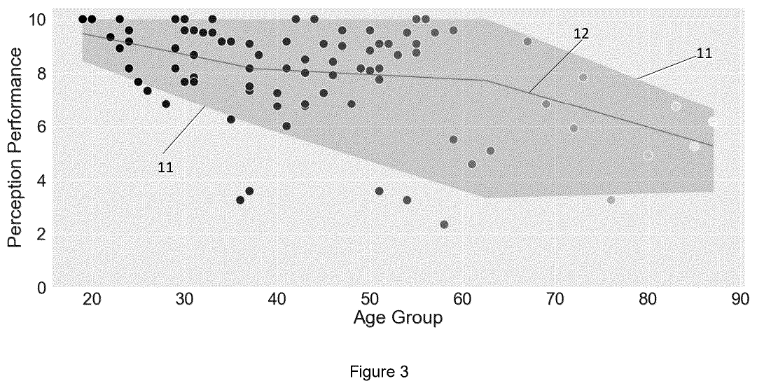

The results for 89 such participants are plotted in FIG. 3. Superimposed on the results is an outline (marked 11) of the anticipated scatter of results by age range derived from other results. It should be noticed that average perception of taste, indicated by the line 12, declines with increasing age. In a medically applied system, outcomes significantly below the normal range for an age group would be taken to indicate further investigation in case the apparent loss of taste and smell perception indicated a problem needing further investigation. This is particularly true in the 40-to-60-year age group, here such results might indicate the beginnings of a neurodegenerative condition such as dementia, which when identified early may be treatable.

Delivery of the samples in the preceding illustrations may be in cups or beakers. However, the samples may be pre-dispensed into vials and supplied to individuals as a test kit, containing the variable samples and an equal number of base samples, with the individual completing the results on a chart as in Table 2 or entering them online though an .app.

An identical approach to that described above can be adopted for smell components, wherein the smell components of a sample are sniffed by a participant and recorded in a similar way to that described above.

Preferably at least 8 comparative pairs are used, with in some cases the comparative flavour being the same as the base flavour. By doing this the chance of an individual successfully guessing correctly all the correct outcomes of the comparison is substantially reduced.

It has now been found that degradation of taste sensation, and/or in the case of brain injury, of smell perception is a possible marker of a brain problem, and the method as described in this invention, among other things, can be used to identify possible early onset of a neurodegenerative condition such as dementia or a brain injury.

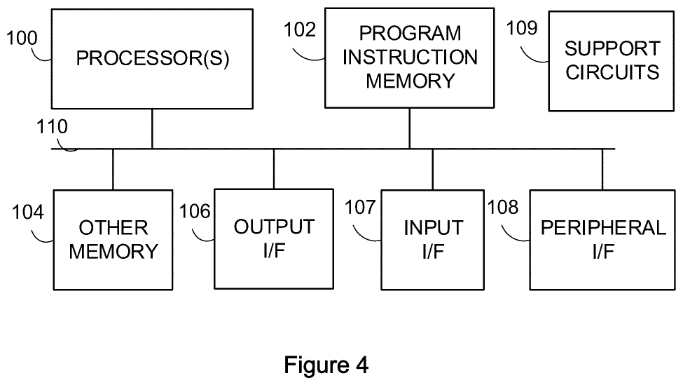

The comparison of an individual's results with those of the previously prepared standards for a person in the same age range may be implemented by electronic machinery. With this in mind, FIG. 4. shows an example of appropriate electronic machinery, e.g., processor circuitry, as comprising one or more processors 100, program instruction memory 102; other memory 104 (e.g., RAM, cache, etc.); input/output interfaces 106 and 107, peripheral interfaces 108; support circuits 109; and busses 110 for communication between the aforementioned units. The processor(s) 100 may comprise the processor circuitries described herein.

A memory or register described herein may be depicted by memory 104, or any computer-readable medium, may be one or more of readily available memory such as random-access memory (RAM), read only memory (ROM), floppy disk, hard disk, flash memory or any other form of digital storage, local or remote, and is preferably of non-volatile nature, as and such may comprise memory. The support circuits 109 are coupled to the processors 100 to support the processor in a conventional manner. These circuits include cache, power supplies, clock circuits, input/output circuitry and subsystems, and the like.

The processes and methods of the disclosed embodiments may be implemented as a software routine. Alternatively, or additionally, some or all of method steps that are disclosed therein may be performed in hardware as well as by a processor running software. As such, the embodiments may be implemented in software, as executed upon a computer system, in hardware as an application specific integrated circuit or other type of hardware implementation, or a combination of software and hardware. The software routines of the disclosed embodiments are capable of being executed on any computer operating system and is capable of being performed using any CPU architecture.

The functions of the various elements including functional blocks, including but not limited to those labelled or described as “computer”, “processor” or “controller”, may be provided through the use of hardware such as circuit hardware and/or hardware capable of executing software in the form of coded instructions stored on computer readable medium. Thus, such functions and illustrated functional blocks are to be understood as being either hardware-implemented and/or computer-implemented, and thus, machine-implemented.

In terms of hardware implementation, the functional blocks may include or encompass, without limitation, digital signal processor (DSP) hardware, reduced instruction set processor, hardware (e.g., digital or analogue) circuitry including but not limited to application specific integrated circuit(s) [ASIC], and/or field programmable gate array(s) (FPGA(s)), and (where appropriate) state machines capable of performing such functions.

In terms of computer implementation, a computer is generally understood to comprise one or more processors or one or more controllers, and the terms computer and processor and controller may be employed interchangeably herein. When provided by a computer or processor or controller, the functions may be provided by a single dedicated computer or processor or controller, by a single shared computer or processor or controller, or by a plurality of individual computers or processors or controllers, some of which may be shared or distributed. Moreover, use of the term “processor” or “controller” may also be construed to refer to other hardware capable of performing such functions and/or executing software, such as the example hardware recited above.

Nodes that communicate using the air interface also have suitable radio communications circuitry. Moreover, the technology disclosed herein may additionally be considered to be embodied entirely within any form of computer-readable memory, such as solid-state memory, magnetic disk, or optical disk containing an appropriate set of computer instructions that would cause a processor to carry out the techniques described herein.

The acts described may be performed by a software program product stored tangibly on a non-transient computer-readable medium which, when executed by one or more processors as herein mentioned, performs such acts either in whole or in part.

Each functional block or various features of the aforementioned embodiments may be implemented or executed by circuitry, which is typically an integrated circuit or a plurality of integrated circuits. The circuitry designed to execute the functions described in the present specification may comprise a general-purpose processor, a digital signal processor (DSP), an application specific or general application integrated circuit (ASIC), a field programmable gate array (FPGA), or other programmable logic devices, discrete gates or transistor logic, or a discrete hardware component, or a combination thereof. The general-purpose processor may be a microprocessor, or alternatively, the processor may be a conventional processor, a controller, a microcontroller or a state machine. The general-purpose processor or each circuit described above may be configured by a digital circuit or may be configured by an analogue circuit. Further, when a technology of making into an integrated circuit superseding integrated circuits at the present time appears due to advancement of a semiconductor technology, the integrated circuit by this technology is also able to be used.

1. A method of indicating degradation of an individual's flavour perception comprising the following acts:
(1) delivering to a user comparative taste sensations via a delivery means, the comparative taste sensations being pairs of artificial flavour sensations to be compared by the individual, each member of each pair comprising a plurality of flavour components selected from the group comprising sweetness, sourness, saltiness, bitterness, umami, astringency, capsaicin, oiliness and, aroma components associated with each flavour of the pair, one flavour within each pair being identical in each of the plurality of pairs, the other member of each pair being the same or different; and
(2) comparing an outcome of the comparison by the individual is against a mean population response to the same comparative flavour sensations and in which deviation below the normal response pattern acts as an indication of degradation of flavour sensation in the individual.

2. A method of indicating degradation of an individual's flavour perception as claimed in claim 1 in which at least eight pairs of artificial flavours are compared by an individual.

3. A method of indicating degradation of an individual's flavour perception as claimed in claim 1 characterised in that the delivery means comprises a plurality of pairs of vials, one vial of a pair containing a flavour that is identical in at least one vial in each of the pairs and the other vial in a pair containing the flavour to be compared by the individual against the flavour that is identical in at least one vial in each of the pairs.

4. A method of indicating degradation of an individual's flavour perception as claimed in claim 3 in which one or more pairs of vials containing flavours to be compared are mounted in a test kit to be supplied to a user.

5. A method of indicating degradation of an individual's flavour perception as claimed in claim 1 used to identify the possible early onset of a neurodegenerative condition.

6. A method of indicating degradation of an individual's flavour perception as claimed in claim 1 used to identify a possible brain injury.

7. A method of indicating degradation of an individual's flavour perception comprising the following acts:
(1) delivering to a user comparative taste sensations via a delivery means, the comparative taste sensations being at least eight pairs of artificial flavour sensations to be compared by the individual, each member of each pair comprising a plurality of flavour components selected from the group comprising sweetness, sourness, saltiness, bitterness, umami, astringency, capsaicin, oiliness and, aroma components associated with each flavour of the pair, one flavour within each pair being identical in each of the plurality of pairs, the other member of each pair being the same or different;
(2) comparing an outcome of the comparison by the individual is compared against a mean population response to the same comparative flavour sensations and in which deviation below the normal response pattern acts as an indication of degradation of flavour sensation in the individual.

8. A method of indicating degradation of an individual's flavour perception as claimed in claim 7 in which the delivery means comprises a at least eight pairs of vials, one vial of a pair containing a flavour that is identical in at least one vial in each of the pairs and the other vial in a pair containing the flavour to be compared by the individual against the flavour that is identical in at least one vial in each of the pairs.

9. A method of indicating degradation of an individual's flavour perception as claimed in claim 8 in which at the least eight pairs of vials containing flavours to be compared are mounted in a test kit to be supplied to a user.

10. A method of indicating degradation of an individual's flavour perception as claimed in claim 7 used to diagnose possible early onset of a neurodegenerative condition.

11. A method of indicating degradation of an individual's flavour perception as claimed in claim 7 used to diagnose a possible brain injury.

12. A method of indicating degradation of an individual's flavour perception as claimed in claim 1, further comprising generating an output indicative of the comparison of act (2).

13. A method of indicating degradation of an individual's flavour perception as claimed in claim 1, further comprising generating an output indicative of the comparison of act (2).

14. A computer program product comprising coded instructions stored on a non-transient medium which, when executed by a processor, perform the acts of claim 1.
